(19) United States
(12) Patent Application Publication
(10) Pub. No.: US 2007/0053985 A1
(43) Pub. Date: Mar. 8, 2007

(54) Coenzyme Q10-containing fine particle with excellent dispersibility

(75) Inventors: Takahiro Ueda, Kobe-shi (JP); Shiro Kitamura, Akashi-shi (JP); Yasuyoshi Ueda, Himeji-shi (JP)

Correspondence Address: SUGHRUE MION, PLLC, 2100 PENNSYLVANIA AVENUE, N.W., SUITE 800, WASHINGTON, DC, 20037, US

(73) Assignee: KANEKA CORPORATION

(21) Appl. No.: 11/509,100

(22) Filed: Aug. 23, 2006

(30) Foreign Application Priority Data
Aug. 24, 2005 (JP) 2005-243143

(51) Int. Cl. A61K 38/43; A61K 9/14; A61K 31/12

(52) U.S. Cl. 424/486; 424/94.1; 514/690

(57) ABSTRACT

The present invention has an object to provide coenzyme Q₁₀-containing particles, which can be easily and simply dispersed into water without any surfactants or other additives, and their production method. The present invention makes it easier to prepare an aqueous dispersion containing coenzyme Q₁₀, which is hard to disperse in water, as fine particles by modifying coenzyme Q₁₀ into a coenzyme Q₁₀-containing fine particle comprising coenzyme Q₁₀ covered with a biocompatible polymer. Therefore, compositions containing dispersed coenzyme Q₁₀ in water, such as drinkable preparations, lotions, etc., can be produced in easy and simple manners.

### BACKGROUND OF THE INVENTION

1. Field of the Invention

The present invention relates to a coenzyme Q₁₀-containing fine particle excellent in dispersibility.

2. Description of the Related Art

Coenzyme Q is an essential component widely distributed in living organisms, from bacteria to mammals. It is known as a constituent of the mitochondrial electron transport system in cells of the living organism. It is known that coenzyme Q functions as a carrier component in the electron transport system through frequent oxidation and reduction in mitochondria. Examples of physiological functions of coenzyme Q include activation of energy production due to mitochondrion-activating effect, activation of cardiac functions, stabilizing effect of cell membranes, protecting effect of cells due to antioxidative effect, and the like. Furthermore, it is known that reduced coenzyme Q shows antioxidative effect.

In human bodies, coenzyme Q₁₀, whose side chain has 10 repetitions of a unit, is a predominant component among the group of coenzyme Q. Coenzyme Q has two different forms including oxidized form and reduced form, and usually about 40 to 90% of coenzyme Q₁₀ is present as reduced form in living organisms. Coenzyme Q is also referred to as “vitamin Q” in view of its vitamin-like functions. It is an ingredient serving as a nutrient source to return weakened cell activities to a healthy state, and thereby can rejuvenate bodies. Among these, coenzyme Q₁₀ is an essential substance to maintain functions of living bodies; it is known as a component locally present in mitochondria, lysosomes, Golgi bodies, microsomes, peroxisome or a cell membrane and involves in ATP production activation, an antioxidative effect in living bodies, and stabilization of membranes as a constituent of an electron transport system. Furthermore, coenzyme Q₁₀ is a compound useful as food, food with nutrient function claims, food for specified health uses, supplements, nutrients, drugs for animals, drinks, feed, cosmetics, drugs, remedies, preventive medicines, etc.

Oxidized coenzyme Q₁₀ is also referred to as “Ubidecarenone”. It is used as health food in Europe and the United States, and also used as medicines for treating congestive-heart-failure in Japan. Recently, it is coming to be used as nutritional food also in Japan.

On the other hand, reduced coenzyme Q₁₀ has a strong antioxidative effect in itself. Therefore, it becomes possible to effectively increase antioxidation activity in blood by sending a sufficient amount of reduced coenzyme Q₁₀ into blood. Such increase of antioxidation activity in blood would be widely effective for prevention of varieties of disorders that would become worse by the action of active oxygens, for example, for prevention of angiopathy on blood return after ischemia, re-stenosis after arteriosclerosis, re-angiopathy after cerebral infarction, arteriosclerosis, or diabetic complication.

However, coenzyme Q₁₀ has a problem in ease of administration, although it shows great effectivenesses and efficacies mentioned above. For example, in case of direct oral administration, coenzyme Q₁₀ causes uncomfortable feelings on going through the throat, and therefore, such direct oral administration has not been accepted commonly. It is conceivable to administer coenzyme Q₁₀ the form of liquid dispersion, such as aqueous dispersion, in order to make it easier to take in coenzyme Q₁₀ powder. However, coenzyme Q₁₀ powder floats on the surface when added to water, and undispersed lumps of particles easily occurs because of its low dispersibility in water. These lumps of particles are not easily crushed by just agitating using a spoon or the like. Therefore, only simple agitation after addition of coenzyme Q₁₀ in water is not enough to produce a drinkable preparation. It is theoretically possible to crush the lumps of particles by powerfully agitating using a homogenizer etc., to approach to a drinkable mixture, but use of such a homogenizer is not a common way for everyone to perform. Even if a mixture of coenzyme Q₁₀ in a liquid were prepared by such a compulsorily agitation, the obtained mixture must not be a drinkable state. Therefore, it is hard to utilize such a mixture as an aqueous dispersion containing dispersed coenzyme Q₁₀ or like compositions. To overcome these problems, and to utilize coenzyme Q₁₀ in the form of such dispersion or the like, it would be necessary to coexist with additives, such as surfactants, etc.

However, it takes great time to select a surfactant suited for coenzyme Q because a variety of surfactants exist. Furthermore, dispersibility may be deteriorated due to the coexistence of other substances in some cases. Moreover, it is known that some specific surfactants cause adverse effects on stability of reduced coenzyme Q₁₀ if reduced coenzyme Q₁₀ is coexisted with such surfactants.

A method for dispersing or emulsify ubiquinone into aqueous solution using water soluble matter is also proposed (Japanese Kokai (unexamined) Publication 2003-55203), but the method is limited in applications because organic acid is required in the method, or the like reasons.

On the other hand, studies of nano-capsules have been made for the purpose of improving sustained release ability of drugs (see Japanese Kokai (unexamined) Publications Hei-5-58882 and Hei-9-110678). However, examples of such nano-capsules using coenzyme Q₁₀ have not been known yet, and technologies for improving dispersibility of such nano-capsules have not been established, either.

### BRIEF SUMMARY OF THE INVENTION

Under the above backgrounds, coenzyme Q₁₀-containing fine particles, which can be easily and simply dispersed into water without any surfactants or other additives, and their production method have been sought for.

Taking the above states into consideration, the present inventors made intensive investigations, and surprisingly, they found that not only dispersibility in water is greatly improved, but also particle characteristics are improved, by covering coenzyme Q₁₀ with a biocompatible polymer. They also found that such covered particles could be processed as compositions including a drinkable preparation, lotion, injectable preparation, etc., since they can be dispersed in water easily and simply without any surfactants. Based on these findings, they have completed the present invention.

Namely, the present invention relates to a coenzyme Q₁₀-containing fine particle, which comprises coenzyme Q₁₀ covered with a biocompatible polymer. Furthermore, the present invention relates to a coenzyme Q₁₀-containing composition, which is obtained by dispersing said coenzyme Q₁₀-containing fine particles in water.

### DETAILED DESCRIPTION OF THE INVENTION

Hereafter, this invention will be explained in detail. In this specification, “coenzyme Q₁₀” is a generic term for representing oxidized coenzyme Q₁₀, reduced coenzyme Q₁₀ and mixture thereof. If simply the term “coenzyme Q₁₀” is used in this specification, it is to be understood as any of oxidized coenzyme Q₁₀, reduced coenzyme Q₁₀, and whole mixture containing both of them.

Oxidized coenzyme Q₁₀ used in this invention can be obtained, for example, by conventional known methods, such as synthesis, fermentation, and extraction from natural products. Especially, one obtained by fermentation, or extraction from natural products is preferred in view of purity, such as content of impurities. Reduced coenzyme Q₁₀ used in this invention can be obtained, for example, by conventionally known methods, such as synthesis, fermentation, and extraction from a natural product. Alternatively, reduced coenzyme Q₁₀ can be also obtained by reduction of oxidized coenzyme Q₁₀ obtained by the above-mentioned methods with reducing agents, such as sodium dithionite, sodium borohydride or ascorbic acid.

The coenzyme Q₁₀-containing fine particles used in this invention comprise coenzyme Q₁₀ and a polymer having biocompatibility, which covers the coenzyme Q₁₀. Namely, said coenzyme Q₁₀ is covered with a polymer that does not have a bad influence on a living body.

The biocompatible polymer used in this invention is not particularly restricted, but preferred is a biodegradable polymer, which does not have bioactivity, but decomposes and disappears in-vivo. Among such a biodegradable polymer, particularly preferred are homopolymers constituted of a hydroxycarboxylic acid, cyanoacrylic acid, trimethylene carbonate, or a ring-opened product of a cyclic lactone; polyethylene glycol, and the like. The hydroxycarboxylic acid, which is a monomeric unit of such polymers, is not particularly restricted, but examples thereof include lactic acid, malic acid, glycolic acid, 3-hydroxypropionic acid, 3-hydroxybutyric acid, 3-hydroxyvaleric acid, 3-hydroxycaproic acid, etc. Preferred are lactic acid and glycolic acid. The cyclic lactone is not particularly restricted, but examples thereof include ε-caprolactone, etc. In the present invention, not only a homopolymer but also a copolymer obtained by co-polymerizing two or more of these monomers can be used. Even a mixture of two or more of such homopolymers and/or copolymers can also be used. Preferable examples of polymers which can be used in this invention include polylactic acid, polyglycolic acid, and a copolymer of lactic acid and glycolic acid [(lactic acid/glycolic acid) copolymer].

Weight average molecular weight of the biocompatible polymer is not particularly restricted. However, lower limit of such weight average molecular weight is generally about 1,000, preferably about 2,000, and more preferably about 5,000, whereas upper limit thereof is generally about 500,000, preferably about 200,000, more preferably about 150,000, and particularly preferably about 100,000. Regarding the copolymer of lactic acid and glycolic acid, monomer proportion between lactic acid and glycolic acid (lactic acid/glycolic acid (mole/mole)) is generally not less than 1/100, preferably not less than 1/10, and more preferably not less than 1/1. The upper limit of the proportion is generally 100/1, preferably 10/1, and more preferably 6/1.

In the present invention, measuring method for the above-mentioned weight average molecular weight depends on the type of biocompatible polymer, and therefore, cannot be restrictedly limited. However, for example, the weight average molecular weight may be determined using the following column and mobile phase in terms of polystyrene equivalent:

Column: Shodex GPC LF-604 (product of Showa Denko K.K.; 6.0 mm (in inside diameter)×150 mm),

Mobile phase: Chloroform

Apparatus for measuring the weight average molecular weight is not particularly restricted, but for example, the weight average molecular weight may be determined using differential refractometer detector (RI) with the above column and mobile phase.

The content of coenzyme Q₁₀ in the coenzyme Q₁₀-containing fine particles covered with a biocompatible polymer is not particularly restricted, but it is generally about 0.01% by weight or more relative to 100% by weight of whole weight of the coenzyme Q₁₀-containing fine particles, and it is preferably about 0.1% by weight or more, more preferably about 0.5% by weight or more, and particularly preferably about 1% by weight or more. On the other hand, it is preferably about 30% by weight or less, preferably about 20% by weight or less, more preferably about 15% by weight or less, and particularly preferably about 10% by weight or less.

The average particle diameter of the coenzyme Q₁₀-containing fine particles covered with the polymer is not particularly restricted, but it may be, for example, about 1 mm or less, preferably about 500 μm or less, more preferably about 100 μm or less, still more preferably about 10 μm or less, particularly preferably about 1 μm or less, and especially preferably about 0.1 μm or less. Needless to say, as the particle diameter is smaller, absorptivity of coenzyme Q₁₀ to a living body will increase.

In the present invention, the particle diameter and the average particle diameter are determined by measuring the particle diameter in an aqueous suspension of obtained coenzyme Q₁₀-containing fine particles, using a laser diffraction-type particle size distribution analyzer. Determination method for the average particle diameter is not particularly restricted, but it is preferred that median size of the particles is used as the average particle diameter.

In the present invention, coenzyme Q₁₀ to be covered with a biocompatible polymer may be any of oxidized form, reduced form, or mixture of these. When it is a mixture, there is no particular restriction for the weight proportion between oxidized coenzyme Q₁₀ and reduced coenzyme Q₁₀. In view of dispersibility to water, comparable effects are expectable regardless of whether the coenzyme Q₁₀ is oxidized form or reduced form. However, reduced coenzyme Q₁₀ is more required to improve fine-particle characteristics than oxidized coenzyme Q₁₀, because reduced form is poor in fine-particle characteristics. For example, it is easy to adhere to containers made of plastics (e.g. polyethylene) or glass. Therefore, for the purpose of improving fine-particle characteristics, it will be more effective to use reduced coenzyme Q₁₀ alone, or mixture of oxidized and reduced form, which contains a certain amount or more of reduced coenzyme Q₁₀, compared to the case where oxidized coenzyme Q₁₀ is used.

What is necessary for the coenzyme Q₁₀-containing fine particles of this invention is that a biocompatible polymer covers core coenzyme Q₁₀, and methods for producing the particles are not particularly restricted. Specific examples of such methods include: (1) a method comprising the step of adding a solution, which contains a biocompatible polymer and coenzyme Q₁₀ in a volatile water-soluble organic solvent, into an aqueous phase with stirring, thereby to precipitate coenzyme Q₁₀-containing fine particles covered with the biocompatible polymer (hereinafter, this method will be referred to as “Method (A)”); (2) a method comprising the step of adding a solution, which contains a biocompatible polymer and coenzyme Q₁₀ in a volatile organic solvent, into an aqueous phase with stirring, thereby to prepare a emulsion or dispersion, followed by evaporation of the organic solvent, to give coenzyme Q₁₀-containing fine particles (hereinafter, this method will be referred to as “Method (B)”); (3) a method comprising the step of spraying a solution, which contains a biocompatible polymer and coenzyme Q₁₀ in a volatile organic solvent, into hot air (hereinafter, this method will be referred to as “Method (C)”); etc. The “volatile organic solvent” as described in this description means a solvent whose boiling point is generally about 120° C. or less, preferably about 100° C. or less, and more preferably about 80° C. in an ordinary pressure (namely, 1 atom (=0.10 MPa)).

Hereinafter, methods of producing coenzyme Q₁₀-containing fine particles covered with a biocompatible polymer will be explained in detail.

As the first method [Method (A)], explained will be a method comprising the step of adding a solution, which contains a biocompatible polymer and coenzyme Q₁₀ in a volatile water-soluble organic solvent, into an aqueous phase, thereby to precipitate coenzyme Q₁₀-containing fine particles covered with the biocompatible polymer.

This is a method of producing desired coenzyme Q₁₀-containing fine particles, which comprises the steps of:

dissolving coenzyme Q₁₀ and a biocompatible polymer in a volatile water-soluble organic solvent; and

adding thus-obtained solution into an aqueous phase with stirring, to precipitate said coenzyme Q₁₀-containing fine particles.

“A water-soluble organic solvent” used here is a solvent with solubility to water of generally not less than about 70% (as volume/volume proportion), preferably not less than about 80%, more preferably not less than about 90%, and particularly preferably not less than about 95%. Most preferred are solvents completely compatible with water. Examples of such water-soluble organic solvents include ketones, alcohols, cyclic ethers, nitrites, etc.

Specifically, ketones containing 3 to 6 carbon atoms are preferable as such ketones, and examples of such ketones include acetone, acetylacetone, etc. As such alcohols, alcohols containing 1 to 5 carbon atoms are preferable, and examples thereof include methanol, ethanol, 1-propanol, 2-propanol, methoxyethanol, ethoxyethanol, etc. Examples of such cyclic ethers include tetrahydrofuran, dioxane, etc. As such nitrites, nitriles containing 2 to 3 carbon atoms are preferable, and examples thereof include acetonitrile, propionitrile, etc. These may be used singly or in combination of two or more of them.

Among them, acetone, methanol, ethanol, 2-propanol and acetonitrile are still more preferred, and acetone and ethanol are particularly preferred among them.

As the aqueous phase, plain water may be used as it is. From the viewpoint to prevent integration of oil drops and/or precipitated fine particles at the time of rapid dispersion or transfer of a solution, which contains coenzyme Q₁₀ and a biocompatible polymer in a water-soluble organic solvent, into aqueous phase, it is preferable to use, for example, an aqueous solution of a dispersant obtained by adding the dispersant in an aqueous phase. Examples of such a dispersant include polyvinyl alcohol, gelatin, gum arabic, starch, carboxymethylcellulose, etc., and polyvinyl alcohol is preferable.

The concentration of such dispersant in the aqueous solution is not particularly restricted, but, for example it is not less than about 0.1 w/w %, preferably not less than about 0.3 w/w %, more preferably not less than about 0.5 w/w %, whereas it is not more than about 10 w/w %, preferably not more than about 5 w/w % and more preferably not more than about 3 w/w %. The value of the above “w/w %” means the percentage of the weight of the dispersant in the whole weight of an aqueous solution.

In the case of using polyvinyl alcohol as a dispersant, polymerization degree of the polyvinyl alcohol is not particularly restricted, but it is generally not less than about 100, preferably not less than about 200, and more preferably not less than about 500. The upper limit of the polymerization degree is generally about 5,000, preferably about 4,000, and more preferably about 3,000. Saponification degree of the polyvinyl alcohol is not particularly restricted, but it is generally not less than about 75, preferably not less than about 80, and more preferably not less than about 85. Needless to say, the upper limit is 100, but preferred are one with the saponification degree of not more than about 98.

Stirring mentioned above can generally be performed using a magnetic stirrer, a mechanical stirrer, etc. It can also be performed using a homogenizer etc.

The coenzyme Q₁₀-containing fine particles covered with a biocompatible polymer can be obtained by separating thus-obtained suspension into solid phase and the liquid phase by centrifugal separation, etc. These coenzyme Q₁₀-containing fine particles can be washed with water if needed. Especially, it is preferable to wash with water when the aqueous solution of a dispersant is used as an aqueous phase.

Thus-obtained coenzyme Q₁₀-containing fine particles covered with a biocompatible polymer may be dried by reduced pressure drying, freeze-drying, through-flow drying, etc., to thereby give a dried form. It is preferred to recover as a dried form from a viewpoint of handleability of fine particles.

The temperature conditions on performing each step, such as dissolution of coenzyme Q₁₀ into an organic solvent, addition of the organic solvent solution into an aqueous phase, separation of suspension, and drying, are not particularly restricted. However, each step can be performed at generally not lower than 0° C., preferably not lower than 10° C., and more preferably not lower than 15° C., whereas it can be performed at generally not higher than 50° C., preferably not higher than 40° C., and more preferably not higher than 30° C. The freeze-drying step can be generally at not higher than −20° C., and preferably not higher than −30° C. Each of these steps can be performed under the ordinary air atmosphere. However, if using only reduced coenzyme Q₁₀ as the coenzyme Q₁₀ component, or using a mixture containing reduced coenzyme Q₁₀ in a higher content, each of these steps can be performed under a deoxidized atmosphere, such as under nitrogen, argon, hydrogen, and carbon dioxide gas in order to prevent conversion of reduced form into oxidized form by air oxidation.

As the second method [Method (B)], explained will be a method comprising the step of adding a solution, which contains a biocompatible polymer and coenzyme Q₁₀ in a volatile organic solvent, into an aqueous phase with stirring, to thereby prepare an emulsion or dispersion, followed by evaporation of the organic solvent, to give coenzyme Q₁₀-containing fine particles.

This method comprises the steps of:

dissolving coenzyme Q₁₀ and a biocompatible polymer in a volatile organic solvent;

adding thus-obtained solution into an aqueous phase with stirring, to prepare an emulsion or dispersion; and

evaporating and removing said volatile organic solvent from said obtained emulsion or dispersion;

to give coenzyme Q₁₀-containing fine particles covered with a biocompatible polymer. In this method, any of water-soluble or insoluble volatile organic solvents may be used. However, this method is preferably applied especially in the case of using a water-insoluble solvent, because this method makes it possible to remove water-insoluble solvents, which are generally hard to transfer into water phase to remove them, in a simple and easy manner.

As the water-soluble organic solvent mentioned here, any of such water-soluble organic solvents as exemplified in the explanation of the above method (A) may be used. A “water-insoluble organic solvent” mentioned here means a solvent having solubility in water of generally not more than about 10 w/w %, and preferably not more than about 5 w/w %. Specific examples thereof include aliphatic or alicyclic hydrocarbons, aromatic hydrocarbons, halogenated hydrocarbons, fatty acid esters, linear ethers, etc.

Examples of such aliphatic or alicyclic hydrocarbons include hexane, cyclohexane, heptane, methylcyclohexane, etc. Examples of such aromatic hydrocarbons include benzene, toluene, etc. Examples of such halogenated hydrocarbons include chloromethane, dichloromethane, chloroform, carbon tetrachloride, chloroethane, chloroethylene, dichloroethylene, etc. Examples of such fatty acid esters include methyl formate, ethyl formate, methyl acetate, ethyl acetate, butyl acetate, etc. Examples of such linear ethers include diethyl ether, isopropyl ether, etc.

Among such solvents, a halogenated hydrocarbon, heptane, hexane, or ethyl acetate are particularly preferred.

The volatile organic solvents may be used singly or in a combination of two or more of them. In the case of using a combination of two or more of such solvents, any of the following combinations may be used: that is, combinations of two or more of water-soluble organic solvents; combinations of two or more of water-insoluble organic solvents; and combinations of any of water-soluble organic solvents and any of water-insoluble ones. In this case, combinations of any of water-soluble organic solvents and any of water-insoluble ones are preferred, and among them, preferred are a combination of any of acetone, methanol, ethanol, 2-propanol and acetonitrile with any of halogenated hydrocarbons, heptane, hexane, and ethyl acetate.

As the aqueous phase, plain water may be used as it is. From the viewpoint to prevent integration of emulsified or dispersed oil drops at the time of rapid dispersion or transfer of a solution, which contains coenzyme Q₁₀ and a biocompatible polymer in a volatile organic solvent, into aqueous phase, it is preferable to use, for example, an aqueous solution of a dispersant obtained by adding the dispersant in an aqueous phase. As such a dispersant, any of such dispersants as exemplified in the explanation of the above method (A) may be used.

Stirring in preparation of emulsion or dispersion can generally be performed using a magnetic stirrer, a mechanical stirrer, etc. It can also be performed using a high-speed homogenizer etc.

Evaporation and removal of the volatile organic solvent used are preferably carried out under a heating or reduced pressure condition, with stirring, for removing the solvent in dispersed droplets. It is more preferred to remove the volatile solvent with heating under a reduced pressure condition in view of removing efficacy.

Thus, suspension containing the coenzyme Q₁₀-containing fine particles covered with a biocompatible polymer can be obtained by evaporating and removing the volatile solvent in such a manner. Further centrifugal separation of the obtained suspension into solid phase and liquid phase gives coenzyme Q₁₀-containing fine particles covered with a biocompatible polymer. The coenzyme Q₁₀-containing fine particles may be washed with water if needed. Especially, it is preferable to wash with water in the case of using an aqueous solution of a dispersant.

Coenzyme Q₁₀ covered with a biocompatible polymer obtained in such a manner can be dried by reduced pressure drying, freeze-drying, through-flow drying, etc., and be obtained as a dried product. Such a dried product is preferred in view of handleability of fine particles.

The temperature conditions on performing each step, such as dissolution of coenzyme Q₁₀ into a volatile organic solvent, addition of the organic solvent solution into an aqueous phase, separation of suspension, and drying, are not particularly restricted. However, each step can be performed at generally not lower than 0° C., preferably not lower than 10° C., and more preferably not lower than 15° C., whereas it can be performed at generally not higher than 50° C., preferably not higher than 40° C., and more preferably not higher than 30° C. The freeze-drying step can be generally at not higher than −20° C., and preferably not higher than −30° C. Each of these steps can be performed under the ordinary air atmosphere. However, if using only reduced coenzyme Q₁₀ as the coenzyme Q₁₀ component, or using a mixture containing reduced coenzyme Q₁₀ in a higher content, each of these steps can be performed under a deoxidized atmosphere, such as under nitrogen, argon, hydrogen, and carbon dioxide gas in order to prevent conversion of reduced form into oxidized form by air oxidation.

As the third method [Method (C)], explained will be a method comprising the step of spraying a solution, which is obtained by dissolving a biocompatible polymer and coenzyme Q₁₀ in a volatile organic solvent, into hot air, to thereby evaporate the volatile organic solvent and give fine particles of coenzyme Q₁₀. The solvent to be used in this method is not particularly restricted provided that it is one of volatile organic solvents that can dissolve the biocompatible polymer.

Examples of such a volatile organic solvent include ketones, alcohols, cyclic ethers, nitriles, aliphatic or alicyclic hydrocarbons, aromatic hydrocarbons, halogenated hydrocarbons, fatty acid esters, linear ethers, etc., which have been mentioned in the explanations of above Method (A) or Method (B).

Coenzyme Q₁₀-containing fine particles covered with a biocompatible polymer can be obtained by spraying this solution containing a biocompatible polymer and coenzyme Q₁₀ in a volatile organic solvent into hot air, in a form of mist with finely dispersing droplets of the solution, to evaporate the volatile organic solvent.

The temperature of the hot air in the method is not particularly restricted, but the method is carried out in a hot air of, for example not lower than about 60° C., preferably not lower than about 80° C., and more preferably not lower than about 100° C.

Three methods were explained, as mentioned above. However, coenzyme Q₁₀-containing fine particles covered with a biocompatible polymer obtained by other methods are also within the scope of the present invention.

The coenzyme Q₁₀-containing fine particles covered with a biocompatible polymer obtained in this manner are easy to be dispersed in water, and well-dispersed aqueous compositions can be prepared without using any surfactants, or the like.

The concentration of the above coenzyme Q₁₀-containing fine particles in the composition dispersed in water depends on the effect or efficacy to be desired for coenzyme Q₁₀, and is particularly restricted. However, it is, for example not less than about 0.01 w/v %, preferably not less than about 0.1 w/v %, more preferably not less than about 1 w/v %, still more preferably about 5 w/v %, and particularly preferably about 10 w/v %. The value of the above “w/v %” expresses a percentage of the weight of the coenzyme Q₁₀-containing fine particles relative to the whole volume of the composition.

The composition of the present invention may further contain another active ingredient. Examples of such an active ingredient include amino acids, vitamins, minerals, polyphenols, organic acids, saccharides, peptides, proteins, etc. A surfactant may be added if needed in order to emulsify and distribute the active ingredient.

Use of the coenzyme Q₁₀-containing fine particle of the present invention makes it possible to prepare a composition containing dispersed coenzyme Q₁₀ in water in a simple and easy manner, without using fats or oils, surfactants, etc., since coenzyme Q₁₀ is covered with a polymer. Therefore, the fine particle is suitable to be used widely in various fields of not only eatables and drinkables such as drinkable preparations, or pharmaceuticals such as injectable preparations, but also cosmetics represented by lotions; thus, it is advantageous. Furthermore, fine-particle characteristics of coenzyme Q₁₀ can be modified, and handleability, transporting ability, etc., are improved by covering coenzyme Q₁₀ with the polymer. Advantageous effects are more efficiently exerted in the case of using reduced coenzyme Q₁₀, which is inherently deficient in fine-particle characteristics.

The method of the present invention makes it easier to prepare an aqueous dispersion containing coenzyme Q₁₀, which is hard to disperse in water. Therefore, compositions containing dispersed coenzyme Q₁₀ in water, such as drinkable preparations, lotions, injectable preparations, etc., can be produced in easy and simple manners, without using fats or oils, surfactants, etc.

### EXAMPLES

Now the present invention will be described further in detail by the following Examples, but the present invention is not limited to these Examples.

Purities of oxidized coenzyme Q₁₀ purities of reduced coenzyme Q₁₀, and weight ratios of reduced coenzyme Q₁₀ and oxidized coenzyme Q₁₀ in Examples are determined by the following HPLC analysis. However, the values of the purities of obtained oxidized coenzyme Q₁₀ or reduced coenzyme Q₁₀ are not the limit values of purities to which the present invention is applicable. Similarly, the weight ratios of reduced coenzyme Q₁₀ relative to total weight of reduced coenzyme Q₁₀ and oxidized coenzyme Q₁₀ should not be understood as the maximum limit value in the present invention.

(HPLC Analysis Conditions)

Column: SYMMETRY C18 (product of Waters), 250 mm (in length), 4.6 mm (in inside diameter),

Mobile-phase: C₂H₅OH:CH₃OH=4:3 (v:v), detection wavelength: 210 nm, flow rate: 1 ml/min, the retention time of reduced coenzyme Q₁₀: 9.1 min, the retention time of oxidized coenzyme Q₁₀: 13.3 min.

### Production Example 1

Into 1000 g of ethanol were added 100 g of oxidized coenzyme Q₁₀ (product of Kaneka Corporation; purity: 99.4%), 60 g of the L-ascorbic acid, and the mixture was stirred at 78° C., to cause reduction reaction. Thirty hours later, the mixture was cooled to 50° C., and 400 g of ethanol and 100 g of water were added in the mixture with maintaining the same temperature. This obtained solution (containing 100 g of reduced coenzyme Q₁₀) was cooled to 2° C. at a cooling rate of 10° C./hour with stirring (required power for stirring: 0.3 kW/m³), and thereby white slurry was obtained. The obtained slurry was filtered under reduced pressure, and resultant wet crystals were washed with cold ethanol, cold water, and cold ethanol subsequently in this order (here, the temperature of cold solvents for washing is 2° C.). Thus-obtained wet crystals were dried under reduced pressure (at 20 to 40° C., 1 to 30 mmHg (=133.32 to 4,000 Pa)), to give 95 g of white dried crystals. In this Production Example 1, all the operations except reduced pressure drying were carried out under the nitrogen atmosphere. The weight ratio of (reduced coenzyme Q₁₀/oxidized coenzyme Q₁₀) in the obtained crystal is 99.5/0.5, and the purity of the reduced coenzyme Q₁₀ is 99.3%.

### Example 1

Oxidized coenzyme Q₁₀ (Product of Kaneka Corporation, consisting of 100% of oxidized form), 140 mg, and (lactic acid/glycolic acid) copolymers [(lactic acid)/(glycolic acid)=1/1 (molar ratio), the weight average molecular weight: 10000], 2800 mg, were added into 80 mL of acetone and then dissolved. This obtained acetone solution was added to 800 mL of 1 w/v % aqueous solution of polyvinyl alcohol (degree of polymerization: 1000) with stirring using a mechanical stirrer, to prepare a suspension. Thus-obtained suspension was subjected to centrifugal separation in a centrifugal separator, to separate fine particles from the suspension. Then, the fine particles were washed with 800 mL of distilled water, followed by freeze-drying the obtained wet fine particles, to give 1900 mg of oxidized coenzyme Q₁₀-containing fine particles covered with the (lactic acid/glycolic acid) copolymer. The content of oxidized coenzyme Q₁₀ in the fine particles is 4.1%, and recovery percentage of oxidized coenzyme Q₁₀ was 58%.

### Example 2

The same operations as Example 1 were carried out except that reduced coenzyme Q₁₀ obtained in Production Example 1 was used instead of oxidized coenzyme Q₁₀. As a result, 1950 mg of reduced coenzyme Q₁₀-containing fine particles covered with (lactic acid/glycolic acid) copolymer were obtained. The content of reduced coenzyme Q₁₀ in a fine particle was 4.0%, and the content of oxidized coenzyme Q₁₀ was 0.1% [the weight ratio of (reduced coenzyme Q₁₀)/(oxidized coenzyme Q₁₀) was 96.5/3.5], and recovery percentage as the total coenzyme Q₁₀ was 59%.

### Example 3

Oxidized coenzyme Q₁₀ (Product of Kaneka Corporation, consisting of 100% of oxidized form), 140 mg, and (lactic acid/glycolic acid) copolymers [(lactic acid)/(glycolic acid)=1/1 (molar ratio), the weight average molecular weight: 10000], 2800 mg, were added into mixed solvent of 30 mL of dichloromethane and 50 mL of acetone, and then dissolved. This obtained solution was added to 800 mL of 1 w/v % aqueous solution of polyvinyl alcohol (degree of polymerization: 1000) with stirring using a mechanical stirrer, to prepare an emulsion. Dichloromethane was evaporated from thus-obtained emulsion under reduced pressure, to prepare a suspension. Thus-obtained suspension was subjected to centrifugal separation in a centrifugal separator, to separate fine particles from the suspension. Then, the fine particles were washed with 800 mL of distilled water, followed by freeze-drying of the obtained wet fine particles, to give 1915 mg of oxidized coenzyme Q₁₀-containing fine particles covered with the (lactic acid/glycolic acid) copolymer. The content of oxidized coenzyme Q₁₀ in a fine particle was 4.1%, and recovery percentage of oxidized coenzyme Q₁₀ was 58%.

### Example 4

The same operations as Example 3 were carried out except that reduced coenzyme Q₁₀ obtained in Production Example 1 was used instead of oxidized coenzyme Q₁₀. As a result, 1915 mg of reduced coenzyme Q₁₀-containing fine particles covered with (lactic acid/glycolic acid) copolymer were obtained. Content of reduced coenzyme Q₁₀ in fine particles was 4.0% and oxidized coenzyme Q₁₀ of the content was 0.1% [the weight ratio of (reduced coenzyme Q₁₀)/(oxidized coenzyme Q₁₀) was 96.5/3.5], and the recovery percentage as the total coenzyme Q₁₀ was 58%.

### Example 5

Oxidized coenzyme Q₁₀ (Product of Kaneka Corporation, consisting of 100% of oxidized form), 150 mg, and (lactic acid/glycolic acid) copolymers [(lactic acid)/(glycolic acid)=1/1 (molar ratio), weight average molecular weight: 10000], 3000 mg, were added into 100 mL of dichloromethane, and then dissolved. This obtained solution was dried by spray-drying with a spray drier (inlet temperature: 170° C.), to give 2800 mg of oxidized coenzyme Q₁₀-containing fine particles covered with the (lactic acid/glycolic acid) copolymer. The content of oxidized coenzyme Q₁₀ in a fine particle was 4.6%, and recovery percentage of oxidized coenzyme Q₁₀ was 86%.

### Example 6

Each 100 mg of the fine particles as obtained in the above Examples 1 to 5 was added into 100 mL of water, and each mixture was then shaken gently. No particles attached to the wall of vessel were shown, and no lumps of particles were occurred. Thus, particles were dispersed easily.

### Example 7

Each 100 mg of the fine particles as obtained in the above Examples 1 to 5 was added into 5 mL of water, and each mixture was then shaken gently. No particles attached to the wall of vessel were shown, and no lumps of particles were occurred. Thus, particles were dispersed easily.

### Comparative Example 1

Oxidized coenzyme Q₁₀ crystals (5 mg) or reduced coenzyme Q₁₀ crystals (5 mg) as obtained in the Production Example 1 was added into 100 mL of water, and each mixture was then shaken gently. Almost all crystals were floated on the water surface, and were not dispersed at all.

### Comparative Example 2

Oxidized coenzyme Q₁₀ crystals (5 mg) or reduced coenzyme Q₁₀ crystals (5 mg) as obtained in the Production Example 1 was added into 100 mL of water, and each mixture was then stirring violently. In the both mixtures, one part of fine particles adhered to the wall of vessel, and another part thereof formed lumps of particles floated on the water surface. Thus, the particles were not dispersed easily.

### Example 8

Fine particles as obtained in Example 4 (500 mg) was placed in a 10-mL glass sample bottle, and then shaken. As a result, almost no particles were adhered to the wall of bottle.

### Comparative Example 3

Reduced coenzyme Q₁₀ crystals as obtained in Production Example 1 (500 mg) was placed in a 10-mL glass sample bottle, and then shaken in the same manner as Example 8. As a result, most of the crystals were adhered to the wall.

### Preparation Example 1

By adding sterile purified water to the mixture of vitamin C, citric acid, sucrose, and the fine particles obtained in the Example 1, a drinkable preparation consisting of the following ingredients was prepared by a conventional method.

| | |
|---|---|
| Vitamin C: | 0.5% by weight |
| Citric acid: | 1.0% by weight |
| Sucrose: | 3.0% by weight |
| Fine particles | 1.0% by weight |
| obtained in the Example 1: | |
| Sterile purified water: | Appropriately added so that |
| | total weight of the drinkable |
| | preparation should be 100.0% by |
| | weight |

### Preparation Example 2

By adding sterile purified water to the mixture of vitamin C, nicotinamide, taurine, citric acid, honey, and the fine particles obtained in the Example 4, a drinkable preparation consisting of the following components was prepared by a conventional method.

| | |
|---|---|
| Vitamin C: | 0.5% by weight |
| Nicotinamide: | 0.03% by weight |
| Taurine: | 1.0% by weight |
| Citric acid: | 1.0% by weight |
| Honey: | 3.0% by weight |
| Fine particles | 1.0% by weight |
| obtained in the Example 4: | |
| Sterile purified water: | Appropriately added so that |
| | total weight of the drinkable |
| | preparation should be 100.0% by |
| | weight |

### Preparation Example 3

By adding sterile purified water to the mixture of squalane, ethanol, glycerol, and the fine particles obtained in the Example 1, a lotion consisting of the following components was prepared by a conventional method.

| | |
|---|---|
| Squalane: | 0.1% by weight |
| Ethanol: | 14.0% by weight |
| Glycerol: | 4.0% by weight |
| Fine particles | 1.0% by weight |
| obtained in the Example 1: | |
| Sterile purified water: | Appropriately added so that |
| | total weight of the lotion |
| | should be 100.0% by weight |

### Preparation Example 4

Sterile purified water was added to the mixture of glucose, Tween 80, and the fine particles obtained in the Example 3, and then pH of the mixture was adjusted by acetic acid. Thus, an injectable preparation consisting of the following components was prepared by a conventional method.

| | |
|---|---|
| Glucose: | 5.0% by weight |
| Tween 80: | 0.3% by weight |
| Fine particles | 1.0% by weight |
| obtained in the Example 3: | |
| Acetic acid (pH adjuster): | Appropriately added |
| Sterile purified water: | Appropriately added so that |
| | total weight of the injectable |
| | preparation should be 100.0% by |
| | weight |

1. A coenzyme Q₁₀-containing fine particle,
which comprises coenzyme Q₁₀ covered with a biocompatible polymer.

2. The coenzyme Q₁₀-containing fine particle according to claim 1,
wherein said biocompatible polymer is a biodegradable polymer.

3. The coenzyme Q₁₀-containing powder according to claim 2,
wherein said biodegradable polymer is at least one polymer selected from the group consisting of polylactic acid, polyglycolic acid, (lactic acid/glycolic acid) copolymer and mixtures thereof.

4. The coenzyme Q₁₀-containing fine particle according to claim 1,
wherein weight average molecular weight of said biocompatible polymer is within the range of 2,000 to 200,000.

5. The coenzyme Q₁₀-containing fine particle according to claim 1,
wherein the average particle diameter of said particle is not more than 1 mm.

6. The coenzyme Q₁₀-containing fine particle according to claim 1,
wherein content of coenzyme Q₁₀ in said particle is 0.01 to 30% by weight.

7. The coenzyme Q₁₀-containing fine particle according to claim 1,
wherein said coenzyme Q₁₀ is reduced coenzyme Q₁₀.

8. A method of producing coenzyme Q₁₀-containing fine particles, which comprises the steps of:
dissolving coenzyme Q₁₀ and a biocompatible polymer in a volatile water-soluble organic solvent; and
adding thus-obtained solution into an aqueous phase with stirring, to precipitate said coenzyme Q₁₀-containing fine particles.

9. The method according to claim 8,
wherein said volatile water-soluble organic solvent is at least one solvent selected from the group consisting of ketones, alcohols, cyclic ethers and nitriles.

10. The method according to claim 8,
wherein the aqueous phase is an aqueous solution of a dispersant.

11. The method according to claim 10,
wherein said dispersant is polyvinyl alcohol.

12. A method of producing coenzyme Q₁₀-containing fine particles, which comprises the steps of:
dissolving coenzyme Q₁₀ and a biocompatible polymer in a volatile organic solvent;
adding thus-obtained solution into an aqueous phase with stirring, to prepare an emulsion or dispersion; and
evaporating and removing said volatile organic solvent from said obtained emulsion or dispersion, to give said coenzyme Q₁₀-containing fine particles.

13. The method according to claim 12,
wherein said volatile organic solvent is at least one solvent selected from the group consisting of aliphatic or alicyclic hydrocarbons, aromatic hydrocarbons, halogenated hydrocarbons, fatty acid esters, linear ethers, ketones, alcohols, cyclic ethers and nitrites.

14. The method according to claim 12,
wherein said volatile organic solvent is a water-insoluble organic solvent.

15. The method according to claim 12,
wherein two or more of volatile organic solvents are used in combination.

16. The method according to claim 12,
wherein evaporation and removal of said volatile organic solvent is carried out under a reduced pressure condition.

17. The method according to claim 12,
wherein the aqueous phase is an aqueous solution of a dispersant.

18. The method according to claim 17,
wherein said dispersant is polyvinyl alcohol.

19. A method of producing coenzyme Q₁₀-containing fine particles, which comprises the steps of:
dissolving coenzyme Q₁₀ and a biocompatible polymer in a volatile organic solvent; and
spraying thus-obtained solution into hot air in a form of mist to evaporate the volatile organic agent and to thereby give said coenzyme Q₁₀-containing fine particles.

20. The method according to claim 19,
wherein temperature of said hot air is not less than 60° C.

21. The method according to claim 19,
wherein said volatile organic solvent is at least one solvent selected from the group consisting of ketones, alcohols, cyclic ethers, nitrites, aliphatic or alicyclic hydrocarbons, aromatic hydrocarbons, halogenated hydrocarbons, fatty acid esters and linear ethers.

22. A coenzyme Q₁₀-containing composition which is obtained by dispersing coenzyme Q₁₀-containing fine particles according to claim 1 in water.

23. The coenzyme Q₁₀-containing composition according to claim 22,
wherein content of said coenzyme Q₁₀-containing fine particles in the composition is within the range of 0.01 to 10% by weight.

24. The coenzyme Q₁₀-containing composition according to claim 22,
which further comprises another active agent than coenzyme Q₁₀.

25. The composition according to claim 22,
which is processed into a drinkable preparation, a lotion, or an injectable preparation.
